(19) United States
(12) Patent Application Publication
(10) Pub. No.: US 2019/0388498 A1
(43) Pub. Date: Dec. 26, 2019

(54) READY-TO-USE OXYTOCIN FORMULATION AND USES THEREOF

(71) Applicant: Eton Pharmaceuticals, Inc., Deer Park, IL (US)

(72) Inventor: Sean E. Brynjelsen, Barrinton, IL (US)

(21) Appl. No.: 16/013,665

(22) Filed: Jun. 20, 2018

(51) Int. Cl. A61K 38/12; A61K 47/18

(52) U.S. Cl. CPC A61K 38/12; A61K 47/183

(57) ABSTRACT

A read-to-use high concentration oxytocin formulation that is free of chlorobutanol.

### FIELD OF THE INVENTION

The present invention is directed to a ready-to-use oxytocin formulation and uses thereof.

### BACKGROUND OF THE INVENTION

Oxytocin is indicated for the initiation or improvement of uterine contractions, where this is desirable and considered suitable for reasons of fetal or maternal concern, in order to achieve early vaginal delivery. It is indicated for various uses, such as: (1) induction of labor in patients with a medical indication for the initiation of labor, such as Rh problems, maternal diabetes, preeclampsia at or near term, when delivery is in the best interests of mother and fetus or when membranes are prematurely ruptured and delivery is indicated; (2) stimulation or reinforcement of labor, as in selected cases of uterine inertia; (3) as adjunctive therapy in the management of incomplete or inevitable abortion. In the first trimester, curettage is generally considered primary therapy. In second trimester abortion, oxytocin infusion will often be successful in emptying the uterus. Other means of therapy, however, may be required in such cases.

Oxytocin is currently available in the United States as a single use 1 ml glass vial. The formulation is typically present at a concentration of 10 United States Pharmacopoeia (USP) Units/ml. The available formulation contains chlorobutanol at 0.5% (5 mg/ml) as a preservative and has a pH of 3.0 to 5.0.

The only approved method of administration of oxytocin for the induction or stimulation of labor is through intravenous (IV) infusion, i.e. the drip method. To prepare the usual solution for infusion, 1-mL Oxytocin Injection, 10 USP Units/mL is combined aseptically with 1,000 mL of non-hydrating diluent (i.e., physiologic electrolyte solution) resulting in Oxytocin concentration of 1 USP Unit/100 mL. The combined solution is then rotated in the infusion bottle to ensure thorough mixing. The infusion bottle typically contains 10 mU/mL. Then, this dilute oxytocic solution is delivered to a patient through use of a constant infusion pump or another similar device to accurately control the rate of infusion.

Thus, one of the drawbacks of the existing oxytocin formulations is that they require multiple mixing steps prior to being administered to a patient, thus increasing the risk of errors, needlestick injuries and possible infections. Furthermore, the container volume for the diluted oxytocin formulation is typically fairly large: 1,000 mL. Typically, however, not more than 250 mL of oxytocin solution fluid is used for medically induced labor, which means that a lot of the diluted oxytocin formulation is currently being wasted.

Another drawback of the existing formulation is that it uses chlorobutanol as a preservative. However, chlorobutanol is a skin irritant and is also highly toxic to the liver. Thus, it would be desirable to avoid chlorobutanol in oxytocin formulations.

Thus, there is a need in the art for a ready-to-use oxytocin formulation which would be free or substantially free of chlorobutanol and could be delivered to a patient without a need for dilution.

### SUMMARY OF THE INVENTION

The present invention is directed to an aqueous ready-to-use formulation comprising oxytocin at a concentration of at least 6 USP Units per 100 mL; saline or dextrose; and wherein the ready-to-use formulation is free or substantially free of chlorobutanol.

In a preferred embodiment, the ready-to-use formulation has a pH of between about 3.0 and about 5.0, and most preferably has a pH of about 3.5.

In a preferred embodiment, oxytocin is present at a concentration of about 6 USP

Units per 100 mL in the read-to-use formulations of the invention.

In one embodiment, the ready-to-use formulation of the invention further contains a stabilizing agent.

In one embodiment, the stabilizing agent comprises sodium edetate.

In another embodiment, the stabilizing agent comprises an amino acid.

In a preferred embodiment, the amino acid is selected from a group consisting of methionine, cysteine and combinations thereof.

In one embodiment, the formulations of the invention include water.

The ready-to-use formulations of the invention may further include other excipients typically used in pharmaceutical formulations, including but not limited to, surfactants, buffers, diluents, and others.

In one embodiment, oxytocin is the only pharmaceutically active compound of the ready-to-use formulations of the invention.

In a preferred embodiment, the formulation exhibits long term stability.

In another embodiment, the invention provides a container comprising the ready-to-use formulation of the invention, wherein the container contains at least 100 mL but not more than 250 mL of the ready-to-use formulation.

In a preferred embodiment, the container contains about 250 mL of the ready-to-use formulation.

In one embodiment, the invention provides a method of medically inducing or stimulating labor, comprising administering to a subject in need thereof an effective amount of the ready-to-use formulation.

In a preferred embodiment, the subject is a mammal.

In a more preferred embodiment, the mammal is a human.

In a preferred embodiment, the administration is done intravenously (IV) or intramuscularly (IM).

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification.

Unless otherwise defined, all technical and scientific terms used herein have the same meaning as commonly understood by one of ordinary skill in the art to which this invention pertains. In the case of conflict, the present document, including definitions will control.

“Around,” “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

Oxytocin is a synthetic polypeptide which has the following chemical structure:

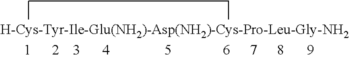

It has a molecular weight of about 1007.2.

The term “oxytocin” as used herein includes both the original nonapeptide having the amino acid sequence as described above, as well as functional analogs such as for example desamino-oxytocin, carbetocin, 4-threonine-1-hydroxy-deaminooxytocin, 9-deamidooxytocin, 7-D-proline-oxytocin and its deamino analog, (2,4-diisoleucine)-oxytocin, 1-desamino-1-monocarba-E12-Tyr (OMe)]-OT(dCOMOT), [Thr⁴-Gly⁷]-oxytocin (TG-OT), the oxytocin agonist as described by Olson et al., (Peptides 12(1): 113-118, 1991), oxypressin and deamino-6-carba-oxytoxin (dC60). Further examples of oxytocin analogues (oxytocics) can for instance be found in MX 9 707 899, disclosing the oxytocin analogues (Mpa¹) oxytocin, (Mpa¹, D-Tyr(Et)², Thr⁴, Orn⁸) oxytocin, (Mpa¹, oxytocin, (Mpa¹, Alae) oxytocin, (Ile²) oxytocin, Gly-(Leu⁴, oxytocin, (D-Asn⁵) oxytocin, (D¹-Cys¹) oxytocin, (Gly⁴) oxytocin, (Leu⁴, D-Arg⁸) oxytocin, (Mpa¹, Leu⁴, D-Arg⁸) oxytocin, (Arg⁸) oxytocin and (Ile⁸) oxytocin.

The term “long-term storage” or “long term stability” is understood to mean that the pharmaceutical composition can be stored for three months or more, for six months or more, and preferably for one year or more, most preferably a minimum stable shelf life of at least two years. Generally speaking, the terms “long term storage” and “long term stability” further include stable storage durations that are at least comparable to or better that the stable shelf typically required for currently available commercial formulations of oxytocin, without losses in stability that would render the formulation unsuitable for its intended pharmaceutical application. Long-term storage is also understood to mean that the pharmaceutical composition is stored either as a liquid at 2-8° C., or is frozen, e.g., at −20° C., or colder. It is also contemplated that the composition can be frozen and thawed more than once.

The term “stable” with respect to long-term storage is understood to mean that oxytocin contained in the pharmaceutical compositions does not lose more than 20%, or more preferably 15%, or even more preferably 10%, and most preferably 5% of its activity relative to activity of the composition at the beginning of storage.

The term “substantially free” means that either no substance is present or only minimal, trace amounts of the substance are present which do not have any substantial impact on the properties of the composition. If reference is made to no amount of a substance, it should be understood as “no detectable amount”.

The term “mammal” includes, but is not limited to, a human.

The term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

The terms “pharmaceutical composition” and “formulation” are used interchangeably.

The present invention is directed to an aqueous ready-to-use formulation comprising oxytocin at a concentration of at least 6 USP Units per 100 mL; saline or dextrose; and wherein the ready-to-use formulation is free of chlorobutanol.

In a preferred embodiment, the ready-to-use formulation has a pH of between about 3.0 and about 5.0, and most preferably has a pH of about 3.5.

In a preferred embodiment, oxytocin is present at a concentration of about 6 USP Units per 100 mL in the read-to-use formulations of the invention.

In one embodiment, the ready-to-use formulation of the invention further contains a stabilizing agent.

In one embodiment, the stabilizing agent comprises sodium edetate.

In another embodiment, the stabilizing agent comprises an amino acid.

In a preferred embodiment, the amino acid is selected from a group consisting of methionine, cysteine and combinations thereof.

In one embodiment, the formulations of the invention include water.

The ready-to-use formulations of the invention may further include other excipients typically used in pharmaceutical formulations, including but not limited to, surfactants, buffers, diluents, and others.

In one embodiment, oxytocin is the only pharmaceutically active compound of the ready-to-use formulations of the invention.

In a preferred embodiment, the formulation exhibits long term stability.

In another embodiment, the invention provides a container comprising the ready-to-use formulation of the invention, wherein the container contains at least 100 mL but not more than 250 mL of the ready-to-use formulation.

In a preferred embodiment, the container contains about 250 mL of the ready-to-use formulation.

In one embodiment, the invention provides a method of medically inducing or stimulating labor, comprising administering to a patient in need thereof an effective amount of the ready-to-use formulation.

In a preferred embodiment, the subject is a mammal.

In a more preferred embodiment, the mammal is a human.

In a preferred embodiment, the administration is done intravenously (IV) or intramuscularly (IM).

What is claimed is:

1. An aqueous ready-to-use formulation comprising oxytocin at a concentration of at least 6 USP Units per 100 mL; saline or dextrose; and wherein the ready-to-use formulation is free or substantially free of chlorobutanol.

2. The ready-to-use formulation of claim 1, wherein the formulation has a pH of between about 3.0 and about 5.0.

3. The ready-to-use formulation of claim 1, wherein the formulation has a pH of about 3.5.

4. The ready-to-use formulation of claim 1, wherein oxytocin is present at a concentration of about 6 USP Units per 100 mL.

5. The ready-to-use formulation of claim 1, wherein the formulation further contains a stabilizing agent.

6. The ready-to-use formulation of claim 5, wherein the stabilizing agent comprises sodium edetate.

7. The ready-to-use formulation of claim 5, wherein the stabilizing agent comprises an amino acid.

8. The ready-to-use formulation of claim 7, wherein the amino acid is selected from a group consisting of methionine, cysteine and combinations thereof.

9. The ready-to-use formulation of claim 1, wherein oxytocin is the only pharmaceutically active compound.

10. The ready-to-use formulation of claim 1, wherein the formulation exhibits long term stability.

11. A container comprising the ready-to-use formulation of claim 1, wherein the container contains at least 100 mL but not more than 250 mL of the ready-to-use formulation.

12. The container of claim 11, wherein the container contains about 250 mL of the ready-to-use formulation.

13. A method of medically inducing or stimulating labor, comprising administering to a subject in need thereof an effective amount of the ready-to-use formulation of claim 1.

14. The method of claim 13, wherein the subject is a mammal.

15. The method of claim 14, wherein the mammal is a human.

16. The method of claim 13, wherein the administering is done intravenously.

17. The method of claim 13, wherein the administering is done intramuscularly.
